(19) United States
(12) Patent Application Publication
(10) Pub. No.: US 2008/0274448 A1
(43) Pub. Date: Nov. 6, 2008

(54) Method for detecting a hydrophobic region of a target substance

(75) Inventors: Hirohiko Tsuzuki, Kanagawa (JP); Masayuki Kawakami, Kanagawa (JP); Masayoshi Yamamoto, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/710,429

(22) Filed: Feb. 26, 2007

(30) Foreign Application Priority Data
Feb. 27, 2006 (JP) 2006-049762

(51) Int. Cl. C12Q 1/00

(52) U.S. Cl. 435/4; 436/86

(57) ABSTRACT

An object of the present invention is to provides a method for detecting a hydrophobic region of a target substance that is capable of forming a hydrophobic region, such as a protein, which can detect a fluorescent analyte with high sensitivity with the use of a simple detection apparatus. The present invention provides a method for detecting a hydrophobic region of a target substance which comprises steps of allowing a target substance capable of forming a hydrophobic region to come into contact with a chemiluminescent substance, and assaying chemiluminescence.

### TECHNICAL FIELD

The present invention relates to a method for detecting a hydrophobic region of a target substance that is capable of forming a hydrophobic region, such as a protein. More particularly, the present invention relates to a method for detecting a hydrophobic region of a target substance with the utilization of chemiluminescence.

### BACKGROUND ART

At present, drug screening is conducted in a wide variety of ways. In recent years, the ATLAS system (Anadys Pharmaceuticals), wherein target proteins are heated to aggregate them and two types of fluorescence-labeled monoclonal antibodies against a given protein epitope are used to perform FRET detection, has been available. Also, a method of fluorescence assay, wherein target proteins are heated to aggregate them and a fluorescent substance, i.e., an allylnaphthanelesulfonic acid, that binds specifically to the exposed hydrophobic region is used to perform fluorescence measurement, has drawn attention. According to such techniques, drug screening is carried out based on a shift toward a higher temperature region upon binding of a drug having a high affinity with a target protein to the target protein. All such techniques employ fluorescence detection methods alone, and no such techniques employ chemiluminescence detection.

However, the fluorescence detection methods as mentioned above, disadvantageously, have low sensitivity, involve the use of a complicated detection apparatus, or are incapable of measurement in the case of a fluorescent analyte.

US Patent Publication 20030059811A1 and U.S. Pat. No. 6,303,322 are cited as a background art.

### DISCLOSURE OF THE INVENTION

The present invention is intended to overcome the drawbacks of the prior art techniques described above. Specifically, an object of the present invention is to provide a method for detecting a hydrophobic region of a target substance that is capable of forming a hydrophobic region, such as a protein, which can detect a fluorescent analyte with high sensitivity with the use of a simple detection apparatus.

The present inventors have conducted concentrated studies in order to attain the above object. As a result, they have found that a hydrophobic region of a target substance can be detected by allowing a target substance capable of forming a hydrophobic region to come into contact with a chemiluminescent substance and then measuring the resulting chemiluminescence. This has led to the completion of the present invention.

The present invention provides a method for detecting a hydrophobic region of a target substance which comprises steps of allowing a target substance capable of forming a hydrophobic region to come into contact with a chemiluminescent substance, and assaying chemiluminescence.

Further, the present invention provides a method for analyzing affinity between a target substance and an analyte, which comprises steps of: (1) incubating a target substance capable of forming a hydrophobic region in the presence of an analyte at a constant temperature; (2) allowing the mixture of the above (1) to come into contact with a chemiluminescent substance; and (3) assaying chemiluminescence.

Further, the present invention provides a method for analyzing affinity between a target substance and an analyte, which comprises steps of: (1) incubating a target substance capable of forming a hydrophobic region in the presence of and in the absence of an analyte at a constant temperature; (2) allowing the mixture of the above (1) to come into contact with a chemiluminescent substance; and (3) assaying chemiluminescence in the presence of and in the absence of an analyte and comparing the assayed chemiluminescence levels.

Preferably, the step (1) comprises preparing a plurality of mixtures of target substances capable of forming hydrophobic regions and analytes and incubating the resulting mixtures at different temperatures.

Preferably, the assayed intensity levels of chemiluminescence are plotted for each of the different incubation temperatures to prepare heat development curves.

Preferably, intermediate temperature (Tm) is determined from the heat development curve obtained in the presence of the analyte and from the heat development curve obtained in the absence of the analyte, and the determined Tm are compared.

Preferably, the target substance capable of forming a hydrophobic region is a protein.

Preferably, the chemiluminescent substance is an adamantane compound.

Preferably, the chemiluminescent substance is disodium 2-chloro 5-(4-methoxyspiro {1,2-dioxetane-3,2′-(5′-chloro)tricyclo[3,3,1,1]decan}-4-yl)phenyl phosphate.

### BRIEF DESCRIPTION OF THE DRAWING

FIG. 1 shows a heat development curve obtained in Example 1.

### BEST MODE FOR CARRYING OUT THE INVENTION

The embodiment of the present invention is described below.

The present invention relates to a method for detecting a hydrophobic region of a protein or the like (a denatured protein) with the utilization of chemiluminescence. In one embodiment of the method according to the present invention, trace amounts of target proteins and inhibitors therefor are added to a microtube or the like, the tube is incubated at arbitrary temperatures, and chemiluminescent substance is added in order to assay the degree of protein denaturation caused by heating at each temperature. The heat-denatured proteins expose hydrophobic region, and chemiluminescent substance binds to the hydrophobic region and emits chemiluminescence. By plotting the intensities of chemiluminescence at each temperature, curves (i.e., heat development curves) are obtained. This procedure is conducted for the case involving the sole use of proteins and for the case involving the use of proteins with inhibitors therefor, and the results thereof are compared to detect changes in the intermediate temperature regions of the curves. Based on the degrees of such changes, the affinity levels of the inhibitors for the target proteins can be ranked.

In the present invention, a chemiluminescent substance is used to detect chemiluminescence generated upon binding of the chemiluminescent substance to a hydrophobic region. Chemiluminescence is a phenomenon whereby molecules excited by a chemical reaction emit energy as light when they return to the ground state. A phenomenon whereby molecules solely produce an excited state is referred to as direct luminescence and a phenomenon whereby molecules transfer energy to a fluorescent agent that is present in the system, following which luminescence of the fluorescent agent is observed, is referred to as indirect chemiluminescence. Bioluminescence is a type of chemiluminescence that utilizes an enzyme reaction.

Specific examples of chemiluminescent substances that can be used in the present invention include luminol compounds and dioxetane compounds. Concerning luminol compounds, it has been known for quite some time that luminol is catalyzed by iron in the blood and emits light, and various studies have been made concerning such phenomenon. Luminol decomposes into an intermediate in the presence of hydrogen peroxide and emits light. This reaction is known to emit strong light upon peroxidase catalysis. In 1985, Kricka et al. discovered that luminol chemiluminescence is increased approximately 1,000 times and the luminescence time is prolonged with the addition of an enhancer, i.e., an iodophenol compound. Kricka et al. designated this method “enhanced chemiluminescence.” The structural formula and a reaction formula of luminol are shown below.

Structural formula of luminol:

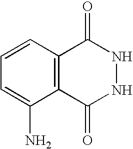

Luminol reaction:

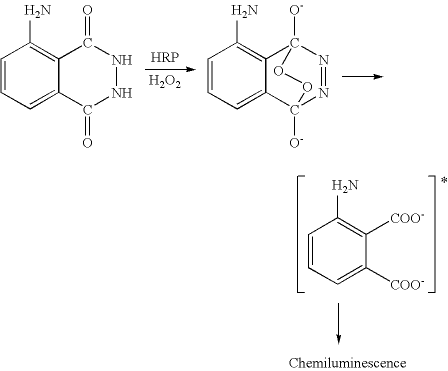

As a dioxetane compound, an alkaline phosphatase substrate (AMPPD), which was reported by Bronstein et al. in 1989, has attracted attention as a chemiluminescent substrate with high sensitivity. Although AMPPD is stable in an aqueous solution, it decomposes into an unstable intermediate compound and emits light when hydrolyzed by alkaline phosphatase. A dioxetane-based substrate has been used for Southern hybridization detection in the field of molecular biology in recent years. CDP-Star, which has higher luminescence intensity than either AMPPD or CSPD, was developed and has spread rapidly. Structural formulae of specific examples of dioxetane compounds and a CDP-Star reaction are shown below. AMPPD, CSPD, and CDP-Star are trademarks or registered trademarks of Tropix, Inc. As chemiluminescent substances used in the present invention, use of adamantane compounds such as AMPPD, CSPD, and CDP-Star mentioned above is preferable.

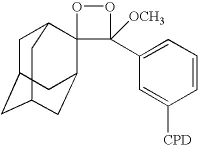

The ImmunoStar Kit commercially available from Wako Pure Chemical Industries, Ltd. can be used for a luminol compound. The Phototope-Star Western Blot detection kit commercially available from Daiichi Pure Chemicals Co., Ltd. or the like can be used for a dioxetane compound. Phototope is a trademark of New England Biolabs.

Chemiluminescence emitted by the chemiluminescent substances mentioned above can be measured using a commercially available Lumino image analyzer (e.g., LAS series, Fuji Photo Film).

In the present invention, any target substances can be used without particular limitation, provided that such substances can form hydrophobic regions. Specific examples of such target substances include peptides, proteins, nucleic acids, sugars, and lipids. Further, liposome, micelles, polymers, grease spots, or the like may be used. The term “protein” refers to a polypeptide having a steric structure, and the number of its amino acids is approximately 20 or more in general. The term “protein” used herein refers to a receptor, an enzyme, an antibody, and other any proteins. A protein may be monomeric or polymeric, and it may be soluble or insoluble in water, with a protein soluble in water being preferable. A single-, double-, or triple-stranded nucleic acid may be employed. A target substance capable of forming a hydrophobic region is preferably a protein capable of forming a secondary, tertiary, or quartic structure via folding, coiling, or torsion.

The term “analyte” used herein refers to a substance that is tested in terms of its affinity with a target substance. Any chemical substances can be used as analytes without particular limitation. Examples thereof include, but are not limited to, low-molecular-weight organic compounds, nucleic acids such as DNA and RNA, and peptides. As analytes, compounds in a compound library or combinatorial library can also be used.

When a target substance capable of forming a hydrophobic region is incubated in the presence of an analyte, a solution that contains an analyte is mixed with a solution that contains a target substance, and the mixture may then be incubated. Mixing and incubation can be carried out in adequate vessels. Examples of vessels that can be used include test tubes, microtubes, vials, cuvettes, wells of multi-well microplates (e.g., 96- or 384-well microplates), and wells of microtiter plates.

In the present invention, it is preferred that heat development curves are obtained for cases involving the presence and the absence of an analyte. In the present invention, shifts in the thus obtained heat development curves can be evaluated to analyze the affinity between a target substance and an analyte (which hereinafter may also be referred to as a “thermal shift assay”).

A thermal shift assay is conducted based on changes in the ligand dependency of the heat development curve of a target substance, such as a protein or nucleic acid. When a target substance is heated to a temperature that exceeds a given range, the target substance is denatured. By plotting the degree of denaturation as a temperature function, the heat development curve of the target substance can be obtained.

It is generally known that a target substance is stabilized upon the binding thereof to an analyte. As a result of stabilization of a target substance by an analyte, more energy (i.e., heat) is required in order to denature the target substance. Accordingly, binding of the analyte to the target substance results in shifting of the heat development curve toward a higher temperature region. With the utilization of such properties, the affinity level of the analyte with the target substance can be determined. A heat development curve shift (i.e., a Tm shift) indicates that the analyte binds to the target substance.

In the present invention, it is preferable to determine intermediate temperatures (Tm) from the heat development curve obtained in the presence of the analyte and from the heat development curve obtained in the absence of the analyte and to compare such determined Tm. Half of the target substance is denatured at the intermediate temperature (Tm). The intermediate temperature (Tm) can be readily determined by a method known in the art.

Alternatively, one entire heat development curve can be compared with the other entire heat development curve by means of computer analysis or the like.

In order to prepare a smooth heat development curve, a sample is heated in a temperature range that is preferably between 1° C. and 20° C., more preferably between 1° C. and 10° C., and further preferably between 1° C. and 5° C. The temperature range within which a sample is heated is preferably between 25° C. and 100° C.

In the present invention, a plurality of samples can be simultaneously heated. When heating samples at discontinuous temperature intervals, the intensity of chemiluminescence can be assayed following each heating step. Alternatively, samples may further be cooled before the intensity of chemiluminescence is assayed following each heating step. Further, samples may be continuously heated to measure the intensity of chemiluminescence during heating.

With the utilization of the method according to the present invention, a lead compound can be identified. After a compound library or a compound combinatorial library is screened by the method of the present invention, a compound that was identified to have a high affinity with a target substance is chemically modified to prepare a second compound library. Subsequently, this second library can be screened by the method of the present invention. Such screening step and step of preparing a new library can be continued until an analyte having a high-affinity Kd value of 10⁻⁴ to 10⁻⁵ M is obtained, for example.

Chemiluminescence can be continuously and simultaneously read for each sample. At a low temperature, all samples exhibit low chemiluminescence levels. As temperature rises, the chemiluminescence level of a sample increases. The heat development curve for a well containing an analyte having high affinity with the target substance exhibits a shift toward a higher temperature region. As a result, a well containing an analyte having high affinity with the target substance emits chemiluminescence that is weaker than chemiluminescence emitted by a well containing no analyte at a temperature higher than Tm of the target molecule in the absence of the analyte.

According to the method of the present invention, two or more analytes can be simultaneously used for a target substance to conduct analysis. Heat development curves can be prepared for a case involving the use of the target substance alone (i.e., without the analyte) and for a case involving the use of the target substance in the presence of two or more analytes. Subsequently, the intermediate temperatures (Tm) are determined for the curves, and one Tm can be compared with the Tm of the other curve, or one entire heat development curve can be compared with the other heat development curve. Thus, the contribution of the use of two or more analytes in combination to the stability of the target substance can be evaluated.

The present invention is described in greater detail with reference to the following examples, although the technical scope of the present invention is not limited thereto.

### EXAMPLE 1

### Reaction Between CA (Carbonic Anhydrase Isozyme II from Bovine Erythrocytes, Sigma; Hereafter Referred to as “CA”) and Acetazolamide (Sigma)

### (1) Preparation of Reagent

A CA solution (1 mg/ml) was prepared using Tris buffer (100 mM tris-hydroxyaminomethane, 150 mM sodium chloride, pH 9.5) and placed on ice.

A commercially available chemiluminescent substance (disodium 2-chloro 5-(4-methoxyspiro {1,2-dioxetane-3,2′-(5′-chloro)tricyclo[3,3,1,1]decan}-4-yl)phenyl phosphate, CDP-Star, ready to use, Roche) was used.

An acetazoleamide (0.02 mM) solution was prepared using Tris buffer containing 1% DMSO.

### (2) Reaction

A 3.3 μM CA solution (30 μl) and 30 μl of Tris buffer were transferred to a microtube for PCR using a micropipette. Such sample was prepared for each temperature (30, 46, 62, 66, 68, 70, 72, 78, 82, and 94° C.). Separately, 30 μl of a 3.3 μM CA solution and 30 μl of a 0.02 mM acetazoleamide solution were transferred to a microtube for PCR using a micropipette. Such sample was prepared for each temperature (30, 46, 62, 66, 68, 70, 72, 78, 82, and 94° C.). Subsequently, a thermal cycler (TaKaRa PCR Thermal Cycler PERSONAL) was used to heat the sample to a temperature of 30° C. for 3 minutes, and the temperature was then held at 25° C. for 1 minute. Thereafter, 30 μl of CDP-Star (ready to use) was added, and the mixture was slowly agitated at room temperature for 15 minutes. Further, 30 μl of 0.36 μg/ml alkaline phosphatase (ORIENTAL YEAST CO., LTD) was added, and the reaction was allowed to proceed at room temperature for 10 minutes. Such reaction procedure was conducted at the following temperatures: 46, 62, 66, 68, 70, 72, 78, 82, and 94° C.

### (3) Detection

After such procedure was completed, samples in the microtubes were transferred to Nunc 245393 F96 Black plate (a polystyrene titer plate), and the intensity of chemiluminescence was assayed using the LAS-1000 pro (Fuji Film).

### (4) Results

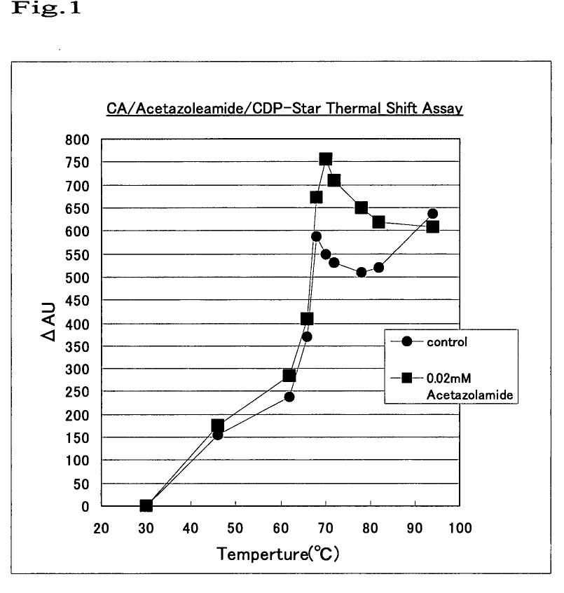

The results are shown in FIG. 1. The vertical axis of the graph shown in FIG. 1 shows values obtained by subtracting the intensity of chemiluminescence at 30° C. from the intensity of chemiluminescence at each temperature. The intermediate temperature determined from the temperature curve of the control sample (protein alone) was 66.3° C., and the intermediate temperature determined from the temperature curve of 0.02 mM acetazolamide was 66.9° C. The intermediate temperature determined from the temperature curve of the protein alone was found to differ from the intermediate temperature determined from the temperature curve representing the case where an inhibitor compound was present. It was also found that the intermediate temperature shifted toward a higher temperature region.

### EXAMPLE 2

The same experiment conducted in Example 1 was conducted using a polystyrene titer plate (SUMILON F Clear, Sumitomo Bakelite Co., Ltd.) instead of the microtiter plate used in Example 1. Results similar to those obtained in Example 1 were obtained.

### COMPARATIVE EXAMPLE 1

A thermal shift assay employing fluorescence detection (with the use of 8-anilino-1-naphthalen-sulfonic acid, ammonium salt hydrate (FW 316.38), ALDRICH) was attempted with the use of a polystyrene titer plate. However, assay could not be performed due to large background noise.

### INDUSTRIAL APPLICABILITY

According to the method for detecting a hydrophobic region of a target substance that is capable of forming a hydrophobic region such as a protein, even a fluorescent analyte can be detected with high sensitivity with the use of a simple detection apparatus.

1. A method for detecting a hydrophobic region of a target substance which comprises steps of allowing a target substance capable of forming a hydrophobic region to come into contact with a chemiluminescent substance, and assaying chemiluminescence.

2. The method according to claim 1, wherein the target substance capable of forming a hydrophobic region is a protein.

3. The method according to claim 1, wherein the chemiluminescent substance is an adamantane compound.

4. The method according to claim 1, wherein the chemiluminescent substance is disodium 2-chloro 5-(4-methoxyspiro {1,2-dioxetane-3,2′-(5′-chloro)tricyclo[3,3,1,1]decan}-4-yl)phenyl phosphate.

5. A method for analyzing affinity between a target substance and an analyte, which comprises steps of: (1) incubating a target substance capable of forming a hydrophobic region in the presence of an analyte at a constant temperature; (2) allowing the mixture of the above (1) to come into contact with a chemiluminescent substance; and (3) assaying chemiluminescence.

6. A method for analyzing affinity between a target substance and an analyte, which comprises steps of: (1) incubating a target substance capable of forming a hydrophobic region in the presence of and in the absence of an analyte at a constant temperature; (2) allowing the mixture of the above (1) to come into contact with a chemiluminescent substance; and (3) assaying chemiluminescence in the presence of and in the absence of an analyte and comparing the assayed chemiluminescence levels.

7. The method according to claim 5, wherein the step (1) comprises preparing a plurality of mixtures of target substances capable of forming hydrophobic regions and analytes and incubating the resulting mixtures at different temperatures.

8. The method according to claim 7, wherein the assayed intensity levels of chemiluminescence are plotted for each of the different incubation temperatures to prepare heat development curves.

9. The method according to claim 8, wherein intermediate temperature (Tm) is determined from the heat development curve obtained in the presence of the analyte and from the heat development curve obtained in the absence of the analyte, and the determined Tm are compared.

10. The method according to claim 5, wherein the target substance capable of forming a hydrophobic region is a protein.

11. The method according to claim 5, wherein the chemiluminescent substance is an adamantane compound.

12. The method according to claim 5, wherein the chemiluminescent substance is disodium 2-chloro 5-(4-methoxyspiro {1,2-dioxetane-3,2′-(5′-chloro)tricyclo[3,3,1,1]decan}-4-yl)phenyl phosphate.
